(19) United States
(12) Patent Application Publication
(10) Pub. No.: US 2020/0306480 A1
(43) Pub. Date: Oct. 1, 2020

(54) HEAD HARNESS FOR A PATIENT INTERFACE, AND PATIENT INTERFACE WITH HEAD HARNESS

(71) Applicant: Loewenstein Medical Technology S.A., Luxembourg (LU)

(72) Inventor: Martin EIFLER, Glueckstadt (DE)

(21) Appl. No.: 16/829,063

(22) Filed: Mar. 25, 2020

(30) Foreign Application Priority Data
Mar. 27, 2019 (DE) 102019107902.0

(51) Int. Cl. A61M 16/06

(52) U.S. Cl. CPC A61M 16/0683

(57) ABSTRACT

Disclosed is a head harness for a patient interface. The head harness is formed from at least one material strand with a reinforcement region and has a first side portion, a second side portion and a base portion. The first side portion and the second side portion of the head harness are each configured for connection to the patient interface, and the base portion connects the first side portion and the second side portion to each other.

### CROSS-REFERENCE TO RELATED APPLICATIONS

The present application claims priority under 35 U.S.C. § 119 of German Patent Application No. 102019107902.0, filed Mar. 27, 2019, the entire disclosure of which is expressly incorporated by reference herein.

### BACKGROUND OF THE INVENTION

### 1. Field of the Invention

The present invention relates to a head harness for a patient interface, wherein the head harness is formed from at least one material strand, wherein the material strand has a reinforcement region at least in some parts.

### 2. Discussion of Background Information

US 2017/0189636 A1, the entire disclosure of which is incorporated by reference herein, discloses headgear for a respiratory mask, having a strap arrangement with front strap portions which can be arranged over a patient's cheeks and whose front ends are configured for attachment to an airway interface portion. The front strap portions have thin stiffening devices which are designed to stiffen, stabilize, position and/or curve the front strap portions.

The stiffening devices help to keep the headgear in an open shape when it is not in use, so as to make it easier for a patient to put on the mask. The stiffening devices are arranged from outside on the front strap portions.

It would be advantageous to have available a harness which permits stabilization of the head harness at least in some parts and which at the same time offers a patient a high degree of wearing comfort.

### SUMMARY OF THE INVENTION

The present invention provides a head harness and by a patient interface as set forth in the independent claims.

Developments and advantageous embodiments are the subject matter of the dependent claims. Further advantages and features will become clear from the general description and from the description of the illustrative embodiments.

The present invention relates to a head harness for a patient interface, wherein the head harness is formed from at least one material strand, wherein the material strand has a reinforcement region at least in some parts.

The present invention also relates to a head harness for a patient interface wherein the material strand has a width, a length and a thickness, the width being always greater than the thickness.

The present invention also relates to a head harness for a patient interface wherein the material strand has a width, a length and a thickness, wherein the reinforcement region provides a reinforcement in such a way that the material strand is less movable in the direction of the width than in the direction of the thickness.

The present invention also relates to a head harness for a patient interface wherein the material strand has a width, a length and a thickness, the reinforcement region being formed in particular along the width and/or length.

The present invention also relates to a head harness for a patient interface wherein the head harness is formed from at least one material strand and has a first side portion, a second side portion and a base portion, the first side portion and the second side portion of the head harness being each configured for connection to the patient interface, and the base portion connecting the first side portion and the second side portion to each other.

The present invention likewise relates to a head harness for a patient interface having a mask with a mask body and a mask bead, wherein the head harness is formed from at least one material strand and has a first side portion, a second side portion and a base portion. The first side portion and the second side portion of the head harness each extend starting from the mask, and the base portion connects the first side portion and the second side portion to each other. The first side portion and the second side portion can each comprise an individual material strand. In an alternative embodiment, the first side portion and the second side portion can each comprise at least two material strands. The first side portion and the second side portion are generally arranged between the base portion and the mask body of the patient interface, the first side portion and the second side portion being each configured to be able to be secured to the patient interface or the mask body of the patient interface. The base portion is generally configured to receive a rear region of a patient's head. Here, the term “receive” means that the base portion or the material strand in the region of the base portion is configured in such a way that it is adaptable to the head shape of a patient. The head harness optionally comprises at least two material strands, generally three material strands. This affords the advantage that, for example, the base portion comprises one material strand, the first side portion comprises at least one material strand, optionally two material strands, and the second side portion likewise comprises at least one material strand, optionally two material strands.

According to the invention, the material strand has at least in some parts a material composition comprising at least a first material layer and a second material layer, an intermediate layer being formed between the first material layer and the second material layer. The intermediate layer is configured as a foam layer. The foam layer is generally covered or enclosed by the first material layer and the second material layer. The first material layer generally has different properties than the second material layer. Generally, at least one of the first material layer and second material layer is made of a material that is kind to skin. Optionally, the respective other material layer is formed from a material that is suitable for the provision of a hook-and-loop fastener. The foam layer generally has a layer thickness of from 1 mm to 5 mm, in particular of from 2 mm to 4 mm.

In one embodiment, the foam layer comprises short fibers and/or long fibers at least in some parts, wherein the short fibers form at least one plane in the foam layer or are distributed over the entire foam layer, the short fibers and/or long fibers being formed in a composite structure. Short fibers have a length of from 0.1 mm to 1 mm. Long fibers have a length of from 1 mm (or greater than 1 mm) to 50 mm. The short fibers and/or the long fibers can be arranged in the foam layer randomly or in an organized manner. Optionally, the long fibers can be fragmented, or they can be reduced into smaller pieces.

The short fibers and/or long fibers are generally formed together in a composite structure in the foam layer. The composite structure can be composed, for example, of a base material, in particular a polyurethane base, in which short fibers or long fibers or a combination of short and long fibers are incorporated. Typically, the composite structure is produced in one production step together with the foam layer. For example, in the production of the foam layer, short fibers and/or long fibers can be mixed with the base material via a mixer head and incorporated directly into the foam layer. A reinforcement of the foam layer is achieved by the direct incorporation of the short fibers and/or long fibers in the composite structure. A thus reinforced region of the material strand can be designated as a reinforcement region or as a stiffening region.

In an alternative embodiment, the material strand comprises at least one structure mat. The structure mat is configured as a reinforcement mat or as a separating mat. Optionally, the structure mat may be configured to separate at least one material layer from the foam layer at least in some parts. A material strand with at least one structure mat forms a reinforcement region. Optionally, the material strand comprises at least one structure mat in parts. This affords the advantage that a reinforcement of the head harness can optionally be provided only in the regions where a reinforcement is required.

In an alternative embodiment, the at least one structure mat is integrated in the intermediate layer. Here, “integrated” can mean, for example, that the structure mat is completely surrounded by the intermediate layer or the foam layer.

For example, the structure mat can be arranged centrally in relation to a layer thickness of the intermediate layer. This embodiment affords the advantage that the structure mat can already be integrated in the intermediate layer during the production of the intermediate layer and is connected to the intermediate layer in a stable manner.

In one development, the foam layer is applied to the structure mat. The structure mat can in this case be designed and configured as a base for the foam layer. In the production of the foam layer, the foam of the foam layer can be applied both to the structure mat and also through the structure of the structure mat or can be incorporated into the structure of the structure mat. In this way, the structure mat is already integrated in the foam layer during the production of the latter.

In an alternative development, the at least one structure mat is arranged between the intermediate layer and the first material layer and/or between the intermediate layer and the second material layer. The intermediate layer or foam layer has a first side and a second side, the first side facing toward the first material layer, and the second side facing toward the second material layer. In the production of the foam layer, in a further step following the formation of the foam layer, the structure mat can thus be applied directly to the first side of the foam layer and/or the second side of the foam layer.

In one development, at least two different structure mats are arranged in or at the intermediate layer. In one embodiment, at least one structure mat is formed at the first side of the foam layer, and at least one structure mat is formed at the second side of the foam layer. Optionally, the structure mat formed at the first side of the foam layer and the structure mat formed at the second side of the foam layer are different. Here, “different” means that the structure mats may comprise different materials, may have different structures, may have been produced by different production processes and/or may have different thicknesses.

In one embodiment, the structure mat is configured as a woven fabric mat, warp-knitted fabric, loop-drawn knitted fabric or braid. Generally, in the embodiment of the material strand with two structure mats, one structure mat has a different structure than the respective other structure mat. For example, a first structure mat can be a woven fabric, while a second structure mat can be a braid or a loop-drawn knitted fabric.

In a further embodiment, the material strand comprises short fibers and/or long fibers and at least one structure mat. This embodiment affords the advantage of a combination of two reinforcement possibilities and the formation of a strong reinforcement region. On the one hand, the short fibers and/or long fibers can be incorporated into the foam layer for the reinforcement. On the other hand, the material strand can be additionally reinforced by at least one structure mat. The structure mat can be integrated in the foam layer or can be formed between the first material layer and the foam layer or between the second material layer and the foam layer.

In one development, the material strand, in the region of the base portion, has a different composition than in the region of the side portions. In this way, the material strand can have, in some parts, a reinforcement by short fibers and/or long fibers or by at least one structure mat. For example, the material strand can have a reinforcement region in the region of the base portion, while the side portions have no reinforcement regions. Alternatively, the side portions may have reinforcement regions, while the base portion has no reinforcement region. Generally, both the base region and the side portions have reinforcement regions at least in some parts.

In one embodiment, the short fibers and/or long fibers and/or the at least one structure mat are or comprise inorganic or organic reinforcement fibers. Inorganic reinforcement fibers can be basalt fibers, boron fibers, glass fibers, ceramic fibers, silica fibers, carbon fibers, quartz fibers and steel fibers. Organic reinforcement fibers can be aramid fibers, carbon fibers, poly(p-phenylene-2,6-benzobisoxazole) (PBO) fibers, polyester fibers, nylon fibers, polyethylene fibers or polymethyl methacrylate fibers. Organic reinforcement fibers can also be natural fibers such as flax fibers, hemp fibers, wood fibers or sisal fibers. Generally, the short fibers and/or long fibers are natural fibers. Alternatively, the short fibers and/or long fibers are synthetic fibers.

The invention further relates to a patient interface comprising an above-described head harness. The patient interface generally comprises a mask with a mask body, a mask bead and the head harness. The patient interface according to the invention affords the advantage that the mask can be held in its position without forehead support. By virtue of the head harness according to the invention, which at least in some parts comprises an above-described material strand, the head harness has at least in some parts a reinforcement that can support and hold the mask on the face or on the head of the patient.

### BRIEF DESCRIPTION OF THE DRAWINGS

Preferred illustrative embodiments of the invention are explained in more detail below with reference to highly simplified schematic drawings, in which:

FIG. 1 shows a perspective plan view of a material strand of a head harness according to the invention,

FIG. 2 shows a side view of the configuration of the material strand shown in FIG. 1,

FIG. 3 shows a side view of a configuration of a material strand of the head harness according to the invention, having an intermediate layer with short fibers,

FIG. 4 shows a side view of a configuration of the material strand of the head harness according to the invention, having an intermediate layer with long fibers,

FIG. 5 shows a side view of a configuration of the material strand of the head harness according to the invention, having an intermediate layer and a structure mat between a first material layer and the intermediate layer,

FIG. 6 shows a side view of a configuration of the material strand of the head harness according to the invention, having an intermediate layer and a structure mat integrated in the intermediate layer,

FIG. 7 shows an embodiment of a patient interface, having a head harness according to the invention fitted in place,

FIG. 8 shows a further embodiment of the patient interface, having a head harness according to the invention fitted in place,

FIG. 9 shows a further embodiment of the patient interface, having a head harness according to the invention fitted in place,

FIG. 10 shows a further embodiment of the patient interface, having a head harness according to the invention fitted in place,

FIG. 11 shows an embodiment of the head harness according to the invention with a reinforcement region.

In the drawings, the same design elements in each case have the same reference numbers.

### DETAILED DESCRIPTION OF EXEMPLARY EMBODIMENTS OF THE INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

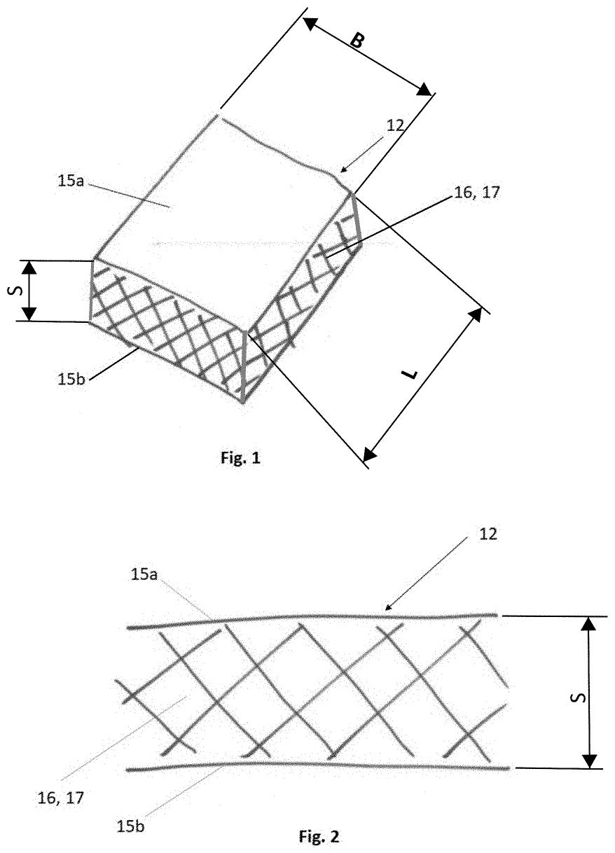

FIG. 1 shows a perspective plan view of a material strand 12 of a head harness 10 according to the invention. The material strand 12 has a width (B), a length (L) and a thickness (S). The width (B) is usually always greater than the thickness (S). The length is usually greater than the width. The material strand 12 has, for example, a reinforcement region. A first material layer 15a and a second material layer 15b are shown, an intermediate layer 16 being formed between the first material layer 15a and the second material layer 15b. The intermediate layer 16, for example, makes up a large part of the thickness (S). The intermediate layer 16 is generally formed, for example, as a foam layer 17.

FIG. 2 shows a side view of the configuration of the material strand 12 shown in FIG. 1. It shows the first material layer 15a and the second material layer 15b, the intermediate layer 16, i.e. the foam layer 17, being formed between the first material layer 15a and the second material layer 15b. The material strand 12 has a width (B), a length (L) and a thickness (S). The width (B) is usually always greater than the thickness (S). The length is usually greater than the width. The material strand 12 has, for example, a reinforcement region.

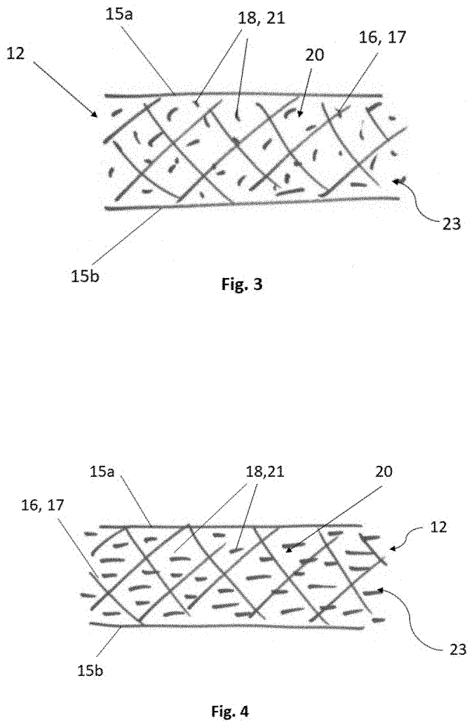

FIG. 3 shows a side view of an embodiment of a configuration of a material strand 12 of the head harness 10 according to the invention, having an intermediate layer 16 with short fibers 18. The first material layer 15a and the second material layer 15b are shown, the intermediate layer 16 being arranged, for example as a foam layer 17, between the first material layer 15a and the second material layer 15b. The short fibers 18 are arranged in the intermediate layer 16.

In the present embodiment, the short fibers 18 are distributed randomly throughout the foam layer 17. In an alternative embodiment, the short fibers 18 may be arranged in the foam layer 17 in an organized manner. In particular, the short fibers 18 may be arranged in the direction of the width B and/or in the direction of the length L. The short fibers 18 are generally formed in a composite structure 20. The composite structure 20 generally comprises a base material in which the short fibers 18 are incorporated. The composite structure 20 is generally incorporated in the foam layer during the production of the foam layer 17. In an alternative embodiment, the short fibers 18 can be arranged only in some parts on or in the foam layer 17. The short fibers 18 can also be configured as long fibers 21. The foam layer 17 and the short fibers 18 in the composite structure 20 form a reinforcement region 23.

FIG. 4 shows a side view of an embodiment of a configuration of the material strand 12 of the head harness 10 according to the invention, having an intermediate layer 16 with long fibers 21. The first material layer 15a and the second material layer 15b are shown, the intermediate layer 16, i.e. the foam layer 17, being formed between the first material layer 15a and the second material layer 15b. The long fibers 21 are arranged in the foam layer 17. In the present embodiment, the long fibers 21 are distributed in an organized manner over the entire surface of the foam layer 17. Alternatively, the long fibers 21 may be arranged randomly in the foam layer 17. In particular, the fibers may be arranged in the direction of the width B and/or in the direction of the length L.

The long fibers 21 are generally formed in the composite structure 20. The composite structure 20 generally comprises the base material into which the long fibers 21 are incorporated. The composite structure 20 is generally incorporated in the foam layer during the production of the foam layer 17. The foam layer 17 and the long fibers 21 in the composite structure 20 form the reinforcement region.

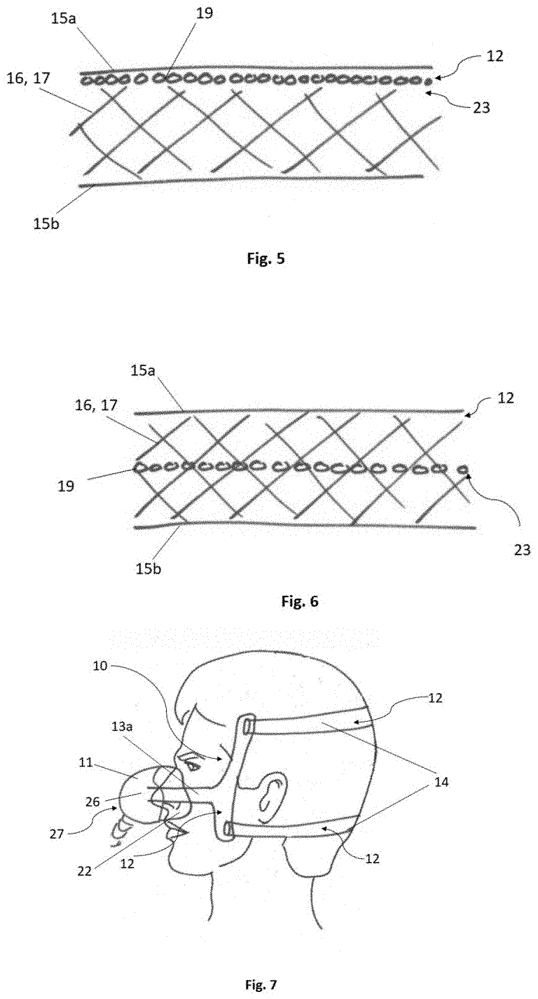

FIG. 5 shows a side view of an embodiment of a configuration of the material strand 12 of the head harness 10 according to the invention, having an intermediate layer 16 and a structure mat 19 between a first material layer 15a and the intermediate layer 16. FIG. 5 shows that the structures 19 are formed in a plane between the intermediate layer 16, or foam layer 17, and the first material layer 15a. Alternatively, the structure mat 19 may be arranged between the second material layer 15b and the intermediate layer 16 or foam layer 17. In an alternative embodiment, at least one structure mat 19 may be arranged in each case between the first material layer 15a and the intermediate layer 16 and between the second material layer 15b and the intermediate layer 16. The foam layer 17 and the structure mat 19 on the foam layer 17 form the reinforcement region 23. The structure mat 19 extends, for example, along the length and/or the width.

FIG. 6 shows a side view of a configuration of the material strand 12 of the head harness 10 according to the invention, having an intermediate layer 16 and a structure mat 19 integrated in the intermediate layer 16. FIG. 6 shows that the structure mat 19 is integrated centrally in the foam layer 17 or intermediate layer 16. Alternatively, the structure mat 19 may be arranged at each position in the intermediate layer 16, i.e. the foam layer 17. In an alternative embodiment, at least two mat structures 19 may be arranged in the intermediate layer 16.

The foam layer 17 and the structure mat 19 in the foam layer 17 form the reinforcement region 23. The structure mat 19 extends, for example, along the length and/or the width.

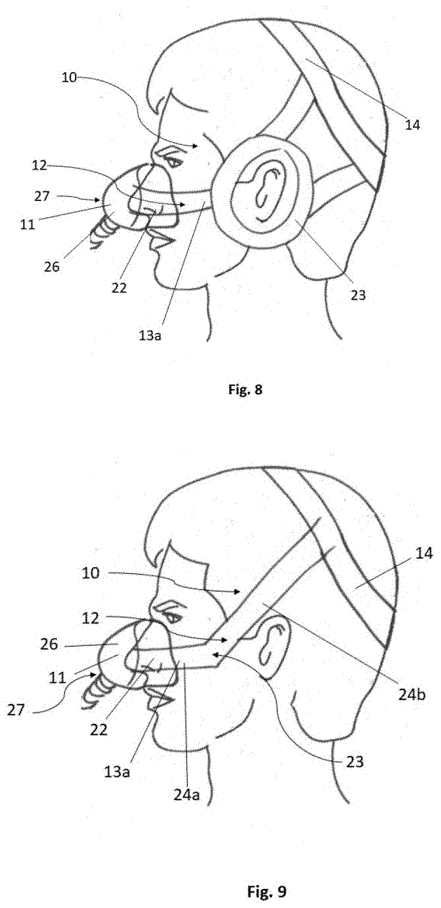

FIG. 7 shows an embodiment of a patient interface 11, having a mask 27 with a mask body 26 and with a mask bead 22 and a head harness 10 according to the invention fitted in place. The head harness 10 according to the invention is shown comprising a first side portion 13a and a base portion 14, the base portion 14 comprising two material strands 12. The first side portion 13a connects the patient interface 11, in particular the mask body 26 of the mask 27, to the base portion 14 of the head harness 10. Both the first side portion 13a and a second side portion 13b (not shown) and the base portion 14 may comprise, at least in some parts, a material strand 12 (shown in FIGS. 1 to 6) which at least in some parts comprises a reinforcement region.

FIG. 8 shows a further embodiment of the patient interface 11, having a mask 27 with a mask body 26 and with a mask bead 22 and a head harness 10 according to the invention fitted in place. The head harness 10 according to the invention is shown comprising the first side portion 13a and the base portion 14. The first side portion 13a has a circular reinforcement region 23, wherein a first material strand 12 of the first side portion 13a is guided to the mask body 26 of the patient interface 11, and a second material strand 12 is guided from the reinforcement region 23 to the base portion 14. Alternatively, the reinforcement region 23 can be oval or polygonal or can have any geometric shape. The reinforcement region 23 comprises a material strand 12, shown in FIGS. 1 to 6. The reinforcement region 23 is formed as a stiffening.

FIG. 9 shows a further embodiment of the patient interface 11, having a mask 27 with a mask body 26 and with a mask bead 22 and a head harness 10 according to the invention fitted in place. FIG. 9 shows the first side portion 13a and the base portion 14. The first side portion 13a has a first part 24a and a second part 24b, the second part 24b being arranged at an angle of from 90° to 180°, in particular of from 45° to 135°, with respect to the first part 24a. The second side portion 13b (not shown) generally has the same configuration. The first side portion 13a generally comprises at least one reinforcement region 23, which is generally formed in a contact region of the first part 24a and of the second part 24b.

The first side portion 13a and the base portion 14 are formed from the material strand 12 according to the invention, the material strand 12 comprising the reinforcement region 23 at least in some parts, in particular in the region of the first side portion 13a and/or of the second side portion 13b. FIG. 9 also shows that the first side portion 13a is arranged at the mask body 26 of the patient interface 11. The second side portion 13b connects the first side portion 13a to the base portion 14. The base portion 14 generally has a substantially circular shape and is designed to at least partially receive a patient's head.

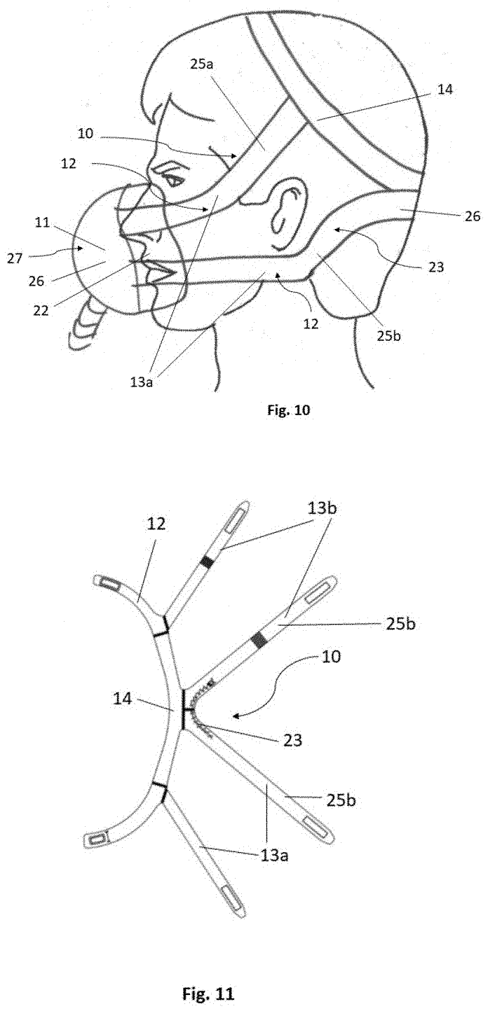

FIG. 10 shows a further embodiment of the patient interface 11, having a mask 27 with a mask body 26 and with a mask bead 22 and a head harness 10 according to the invention fitted in place. FIG. 10 shows the first side portion 13a and the base portion 14. The first side portion 13a is formed from two material strands 12. An upper material strand 25a connects the mask body 26 of the patient interface 11 to the base portion 14. The lower material strand 25b likewise connects the mask body 26 of the patent interface 11 to the base portion 14, the lower material strand 25b being formed separately from the upper material strand 25a. The upper material strand 25a has a curved shape, wherein the upper material strand 25a may comprise a reinforcement region 23 at least in some parts. The lower material strand 25b comprises an arched region 26. The arched region 26 is generally formed of the lower material strand 25b of the first side portion 13a and of the lower material strand 25b of the second side portion 13b. Generally, the arched region 26 comprises one of the material strands 12 (shown in FIGS. 1 to 6) which forms a reinforcement region 23.

FIG. 11 shows the embodiment, shown in FIG. 10, of the head harness 10 according to the invention with the reinforcement region 23. The head harness 10 according to the invention comprises the material strand 12 with the composition according to the invention. The head harness 10 comprises the base portion 14, and also the first side portion 13a and the second side portion 13b. The lower material strand 25b has a reinforcement region 23 in the arched region 26. The reinforcement region 23 in the arched region 26 of the lower material strand 25 affords the advantage that, when the head harness 10 is fitted in place, with the head harness 10 drawn open, the arched region 26 in the reinforcement region 23 is more rigid than the surrounding material strand 12.

### LIST OF REFERENCE NUMERALS

10 head harness
11 patient interface
12 material strand
13a first side portion
13b second side portion
14 base portion
15a first material layer
15b second material layer
16 intermediate layer
17 foam layer
18 short fibers
19 structure mat
20 composite structure
21 long fibers
22 mask bead
23 reinforcement region
24a first part
24b second part
25a upper material strand
25b lower material strand
26 mask body
27 mask

What is claimed is:

1. A head harness for a patient interface, wherein the head harness is formed from at least one material strand, which material strand comprises a reinforcement region at least in some parts thereof.

2. The head harness of claim 1, wherein the reinforcement region is secured to the material strand.

3. The head harness of claim 1, wherein the reinforcement region is integrated in the material strand.

4. The head harness of claim 1, wherein the reinforcement region comprises short fibers and/or long fibers and/or a structure mat and/or a composite structure.

5. The head harness of claim 1, wherein the material strand has a width (B), a length (L) and a thickness (S), the width (B) being always greater than the thickness (S).

6. The head harness of claim 1, wherein the material strand has a width (B), a length (L) and a thickness (S), the reinforcement region providing a reinforcement in such a way that the material strand is less movable in a direction of the width than in a direction of the thickness (S) and/or the reinforcement region being formed in particular along the width (B) and/or the length (L).

7. The head harness of claim 1, wherein the material strand comprises a first material layer and a second material layer, an intermediate layer being arranged between the first material layer and the second material layer and making up the greatest part of a thickness (S).

8. The head harness of claim 7, wherein the intermediate layer comprises or forms the reinforcement region and/or is a foam layer.

9. The head harness of claim 7, wherein the intermediate layer comprises short fibers and/or long fibers at least in some parts, the short fibers forming at least one plane in the intermediate layer or being distributed over the entire intermediate layer, and wherein the short fibers and/or long fibers are formed in a composite structure.

10. The head harness of claim 1, wherein the material strand comprises at least one structure mat.

11. The head harness of claim 10, wherein the at least one structure mat is integrated in an intermediate layer formed between a first material layer and a second material layer of the material strand.

12. The head harness of claim 10, wherein a foam layer is applied to the structure mat.

13. The head harness of claim 10, wherein the at least one structure mat is arranged between an intermediate layer and a first material layer of the material strand and/or between the intermediate layer and a second material layer of the material strand, the intermediate layer being arranged between the first material layer and the second material layer of the material strand.

14. The head harness of claim 10, wherein at least two different structure mats are arranged in an intermediate layer which is formed between a first material layer and a second material layer of the material strand and/or wherein the structure mat is configured as a woven fabric mat, warp-knitted fabric, loop-drawn knitted fabric or braid.

15. The head harness of claim 1, wherein the material strand comprises short fibers and/or long fibers and at least one structure mat.

16. The head harness of claim 15, wherein the short fibers and/or the long fibers and/or the at least one structure mat are or comprise inorganic or organic reinforcement fibers.

17. The head harness of claim 1, wherein the head harness is formed from at least one material strand and comprises a first side portion, a second side portion and a base portion, the first side portion and the second side portion of the head harness being each configured for connection to a patient interface, and the base portion connecting the first side portion and the second side portion to each other.

18. The head harness of claim 17, wherein the first side portion and the second side portion each comprise a reinforcement region and/or the base portion comprises a reinforcement region.

19. A patient interface with a head harness, wherein the patient interface is configured as a mask with a mask body and a mask bead, and the head harness is formed from at least one material strand and has a first side portion, a second side portion and a base portion, the first side portion and the second side portion of the head harness each extending starting from the mask body, and the base portion connecting the first side portion and the second side portion to each other, and wherein the material strand comprises at least in some parts a material composition comprising at least a first material layer and a second material layer, an intermediate layer being formed between the first material layer and the second material layer and being configured as a foam layer.

20. A patient interface, wherein the patient interface comprises the head harness of claim 1.
